(19) United States
(12) Patent Application Publication
(10) Pub. No.: US 2025/0255646 A1
(43) Pub. Date: Aug. 14, 2025

(54) TROCAR ASSEMBLY

(71) Applicant: CILAG GMBH INTERNATIONAL, ZUG (CH)

(72) Inventors: Uri BARAK, Nesher (IL); Roy ZOHAR, Akko (IL); Shai NACHMIAS, Nahariya (IL); Hagay BOTANSKY, Haifa (IL)

(21) Appl. No.: 19/111,455

(22) PCT Filed: Sep. 13, 2023
(86) PCT No.: PCT/IL2023/050999

§ 371 Date: Mar. 13, 2025

Related U.S. Application Data
(60) Provisional application No. 63/409,231, filed on Sep. 23, 2022.
(60) Provisional application No. 63/409,229, filed on Sep. 23, 2022.

(51) Int. Cl. A61B 17/34; A61B 17/00

(52) U.S. Cl. CPC A61B 17/3494; A61B 17/00234; A61B 2017/347

(57) ABSTRACT

There is provided a kit for minimally invasive surgery, comprising: a trocar cannula, and a depth limiter component comprising: a lumen sized and shaped to accommodate a trocar cannula, wherein an external diameter of the trocar cannula is sized to correspond to an internal diameter of the lumen, a locking mechanism that sets the trocar cannula in a fixed position within the lumen, a housing that includes the lumen and the locking mechanism, and at least one aperture of the housing in fluid communication between an internal cavity of the housing and an external environment external to the housing

### RELATED APPLICATIONS

This application claims the benefit of priority of U.S. Provisional Patent Application No. 63/409,231 filed on Sep. 23, 2022, and of U.S. Provisional Patent Application No. 63/409,229 filed on Sep. 23, 2022 the contents of which are incorporated herein by reference in their entirety.

### FIELD AND BACKGROUND OF THE INVENTION

The present invention, in some embodiments thereof, relates to medical devices for minimally invasive surgery and, more particularly, but not exclusively, to a trocar assembly.

Trocars are used during minimally invasive surgery, such as laparoscopic surgery. Trocars are placed through the skin of the body of a subject into a cavity, for example, abdomen or chest. The trocars provide access points into the cavity for tools such as graspers, scissors, staplers, and cameras.

### SUMMARY OF THE INVENTION

According to a first aspect, a kit for minimally invasive surgery, comprises: a trocar cannula, and a depth limiter component comprising: a lumen sized and shaped to accommodate a trocar cannula, wherein an external diameter of the trocar cannula is sized to correspond to an internal diameter of the lumen, a locking mechanism that sets the trocar cannula in a fixed position within the lumen, a housing that includes the lumen and the locking mechanism, and at least one aperture of the housing in fluid communication between an internal cavity of the housing and an external environment external to the housing.

In a further implementation form of the first aspect, the locking mechanism comprises a pressure element designed to apply mechanical pressure for setting the trocar cannula in the fixed position by friction.

In a further implementation form of the first aspect, the locking mechanism comprises a lever connected to a C shaped pin, the C shaped pin designed to engage the trocar cannula and apply mechanism pressure to the trocar cannula in response to a pressure applied to the lever.

In a further implementation form of the first aspect, the locking mechanism comprises a movable protruding element designed to engage an indentation of the trocar cannula.

In a further implementation form of the first aspect, the depth limiter further includes a distal surface that is substantially flat and smooth, for contacting skin of a subject surrounding an entry point of the trocar cannula.

In a further implementation form of the first aspect, the depth limiter further includes a tapered outer surface that is substantially smooth, that tapers outwards from an edge of a distal surface.

In a further implementation form of the first aspect, the depth limiter further comprises a smooth round surface for connecting between an edge of the distal surface and the tapered outer surface.

In a further implementation form of the first aspect, the cavity of the housing is located in a thickness between the lumen and an external lateral surface.

In a further implementation form of the first aspect, the at least one aperture of the housing comprises at least one first aperture located on a distal surface of the housing and at least one second aperture located on a proximal surface of the housing.

In a further implementation form of the first aspect, at least one of the first aperture and the second aperture are arc-shaped.

In a further implementation form of the first aspect, the housing comprises at least one third aperture located along a surface of the housing forming the lumen, the at least one third aperture in fluid communication with the at least one aperture of the housing.

In a further implementation form of the first aspect, moving components of the locking mechanism are disposed within the internal cavity of the housing and in fluid communication with the external environment.

In a further implementation form of the first aspect, the locking mechanism is reversible, for releasing the trocar cannula from the fixed position, for at least one of: removal of the trocar cannula from the lumen, and insertion of the trocar cannula further into the lumen.

In a further implementation form of the first aspect, dimensions of the depth limiter are selected for maintaining a trocar cannula in a substantially upright position that is approximately perpendicular to skin of a subject, while the trocar cannula is fixed in position in the depth limiter, and when the trocar cannula is not being supported by an external entity.

In a further implementation form of the first aspect, the dimensions of the depth limited are selected for maintaining an instrument in the substantially upright position that is approximately perpendicular to skin of the subject, when the instrument is inserted through the trocar cannula piercing the skin into the body of the subject, and when the instrument is not being supported by the external entity.

In a further implementation form of the first aspect, the depth limiter component is made from a biocompatible material that is resistant to autoclaving, designed for multi-use.

In a further implementation form of the first aspect, the lumen includes a first guide element set for engaging a corresponding second guide element of the trocar cannula, wherein the trocar cannula is insertable into the lumen by engaging the first guide element with the second element, and the trocar cannula is prevented from being inserted into the lumen when the first guide element does not engage the second guide element.

In a further implementation form of the first aspect, the first guide element engaging the second guide element prevent rotation of the trocar cannula within the depth limiter.

In a further implementation form of the first aspect, further comprising a plurality of depth limiters having lumens with a plurality of different diameters for accommodating trocar cannulas with different diameters.

In a further implementation form of the first aspect, further comprising a trocar sleeve that includes a trocar housing and the trocar cannula.

In a further implementation form of the first aspect, further comprising a sealing element designed to engage the trocar housing.

According to a second aspect, a method of performing minimally invasive surgery, comprises: passing a trocar cannula into a body of a subject via a lumen of a depth limiter, and activating a locking mechanism of the depth limiter for setting the trocar cannula at a selected fixed position, wherein instruments for treating tissue in the body of the patient are passed via the trocar cannula secured at the selected fixed position by the locking mechanism of the depth limiter.

In a further implementation form of the second aspect, further comprising releasing the locking mechanism, and at least one of: adjusting the position of the trocar cannula relative to the depth limiter and resetting the locking mechanism, and removing the trocar cannula from the lumen of the depth limiter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic of a trocar assembly that includes a depth limiter, in accordance with some embodiments of the present invention;

FIG. 2 is a perspective view of depth limiter, in accordance with some embodiments of the present invention;

FIG. 3 is a side view of depth limiter, in accordance with some embodiments of the present invention;

FIG. 4 is a top view of depth limiter, in accordance with some embodiments of the present invention;

FIG. 5 is a bottom view of depth limiter, in accordance with some embodiments of the present invention;

FIG. 6 is a cross sectional view of depth limiter, in accordance with some embodiments of the present invention;

FIG. 7 is an exploded view of depth limiter, in accordance with some embodiments of the present invention;

FIG. 8 is a schematic of an exemplary sealing element of trocar assembly, in accordance with some embodiments of the present invention;

FIG. 9 is a schematic of an exemplary obturator of trocar assembly, in accordance with some embodiments of the present invention; and

FIG. 10 is a flowchart of an exemplary method of treating a subject using a depth limiter, in accordance with some embodiments of the present invention.

### DESCRIPTION OF SPECIFIC EMBODIMENTS OF THE INVENTION

The present invention, in some embodiments thereof, relates to medical devices for minimally invasive surgery and, more particularly, but not exclusively, to a trocar assembly.

As used herein, the terms proximal and distal are used with reference to a user using the trocar assembly on a subject. Proximal refers to the portions of the trocar assembly that are closer to the user during use. Distal refers to the portions of the trocar assembly that are further away from the user during use.

An aspect of some embodiments of the present invention relates to a depth limiter component designed to engage with a trocar cannula, for performing minimally invasive surgery, for example, laparoscopic surgery on an abdomen, chest, joint, and the like. The depth limiter component includes a lumen sized and shaped to accommodate the trocar cannula. The trocar cannula may be pushed through the lumen to a desired depth though the skin and/or into the body of the subject, which corresponds to a selected length of the trocar cannula that is passed though the lumen. The depth limiter includes a locking mechanism that sets the trocar cannula at the selected length. The locking mechanism fixes the position of the trocar cannula at a fixed position relative to the lumen of the depth limiter.

A housing of the depth limiter includes the lumen and the locking mechanism. Optionally, the housing includes one or more apertures that provide fluid communication between an internal cavity of the housing and an external environment external to the housing. The aperture(s) enable washing fluid to enter the internal cavity for washing out debris from the internal cavity to the external environment. The aperture(s) enable sterilization of the internal cavity, which allows the depth limiter to undergo multiple washing and sterilization cycles, for re-use in multiple surgical procedures.

An aspect of some embodiments of the present invention relates to a kit that includes the depth limiter and at least one additional component, for example, another depth limiter of a same lumen diameter, another depth limiter of another lumen diameter, and a trocar cannula having an external diameter corresponding to the internal diameter of the lumen of the depth limiter.

An aspect of some embodiments of the present invention relates to a method of performing minimally invasive surgery, for example, laparoscopic surgery on an abdomen of a subject. A trocar cannula is passed into a body of a subject via a lumen of a depth limiter. A locking mechanism of the depth limiter is set for setting the trocar cannula at a selected fixed position relative to the lumen of the depth limiter. Instruments for treating tissue in the body of the patient are passed via the trocar cannula secured at the selected fixed position by the locking mechanism of the depth limiter.

The depth limiter, method of treatment, and/or other components of the kit designed to engage with the depth limiter (e.g., trocar cannula) described herein address the technical problem of trocar cannula penetrating too deeply into the body of the subject. The trocar cannula may come into undesired contact with internal tissues. Such undesired contact may have dangerous consequences, for example, perforation of intestines (e.g., large colon, stomach). Even if the trocar cannula does not come in undesired contact with internal tissues, the trocar cannula may inadvertently adjust its position proximally (e.g., deeper into the body of the subject) or distally (e.g., withdrawing away from the body of the subject), which may make performing operations using instruments passed through the cannula more difficult.

The depth limiter, method of treatment, and/or other components of the kit described herein improve upon prior approaches, where no depth limiter is used. In such approaches, control of depth of the trocar cannula may be done manually by the user, without assistance of the depth limiter.

The depth limiter, and/or method of treatment, and/or other components of the kit described herein address the above discussed technical problem, and/or improve upon the above discussed prior approaches. The depth limiter described herein enables fixing the location of the trocar cannula, which prevents inadvertent slippage of the trocar cannula, further into the body of the subject which may perforate organs, and/or out of the body of the subject which may contaminate the tool. The user may select the depth of the trocar cannula insertion into the body of the subject, and fix the trocar cannula at the selected depth via the locking mechanism.

In at least some embodiments, the locking mechanism of the depth limiter is designed to be activated and/or released single-handedly, for example, with a single finger, by pressing a lever. This allows for the depth limiter to be activated with one hand, while the user (e.g., surgeon) hold the trocar cannula with the other hand (the cannula may be pre-loaded into the depth limiter).

The depth limiter, method of treatment, and/or other components of the kit designed to engage with the depth limiter (e.g., trocar cannula) described herein address the technical problem of the requirement to provide continuous support to the trocar optionally with instruments that are passed into the body of the subject via the trocar cannula penetrating the skin of the subject. The trocar and/or instruments, for example, a camera, scissors, a grabber, and a stapler, which are placed at the distal end of a long rod, need to be held as long as they are in the body via the cannula. Letting go of the trocar and/or instrument causes the trocar and/or instrument to fall to the side, potentially becoming contaminated and/or potentially harming internal tissue. The trocar and/or instrument requires continuous support, for example, by a human user holding the trocar and/or instrument, and/or attachment to a surgical robotic arm. When the trocar and/or instrument cannot be supported, the instrument is removed from the body of the subject.

Dimensions of the depth limiter may be selected for maintaining the trocar assembly in a substantially upright position, optionally when the body cavity (e.g., abdomen) has been inflated. The trocar assembly with depth limiter placed thereon is maintained in the upright position while the trocar cannula is piercing the skin and in the body of the subject. The depth limiter maintains the trocar assembly in a substantially upright position, when the trocar assembly is not being supported by an external entity such as a human operator and/or robotic arm. Optionally, the depth limiter is sized and/or shaped for maintaining the trocar assembly in a substantially upright position while an instrument is passed through the trocar assembly. The instrument is located at the distal end region of a long rod that is passed into the body of the subject via the trocar cannula. Without the depth limiter, letting go of the trocar assembly results in the trocar assembly falling from the previously held substantially upright position to the side. The tilt in the trocar assembly to the side may damage internal organs, and/or place the instrument located in the trocar assembly outside of a sterile field which may contaminate the instrument. The tilt to the side of the trocar cannula may damage the skin and/or other tissues at the point of access into the body.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

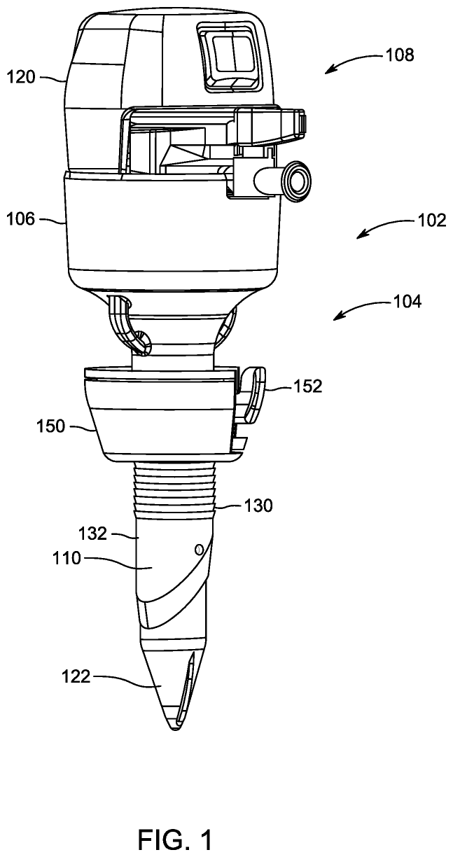

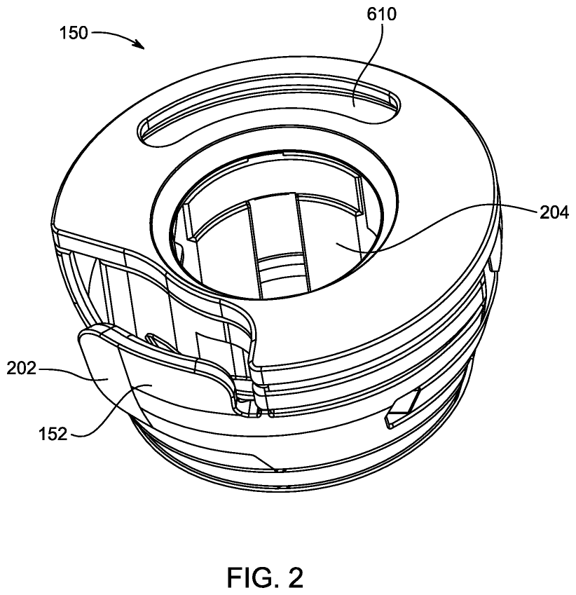

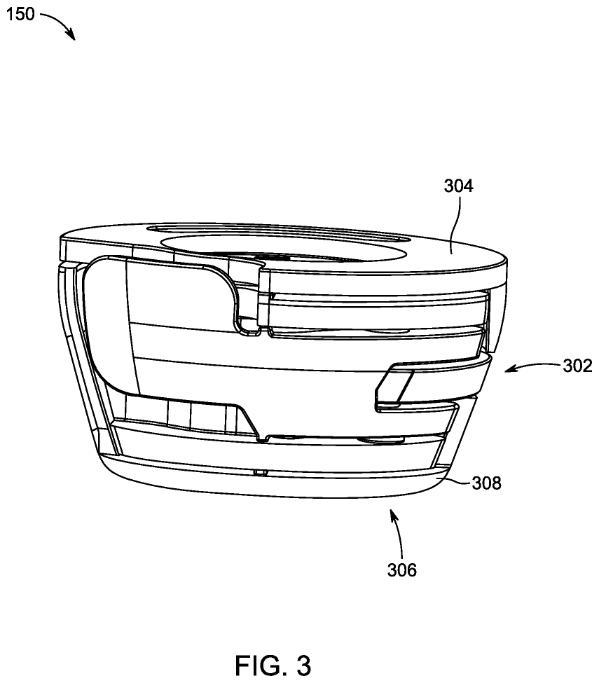

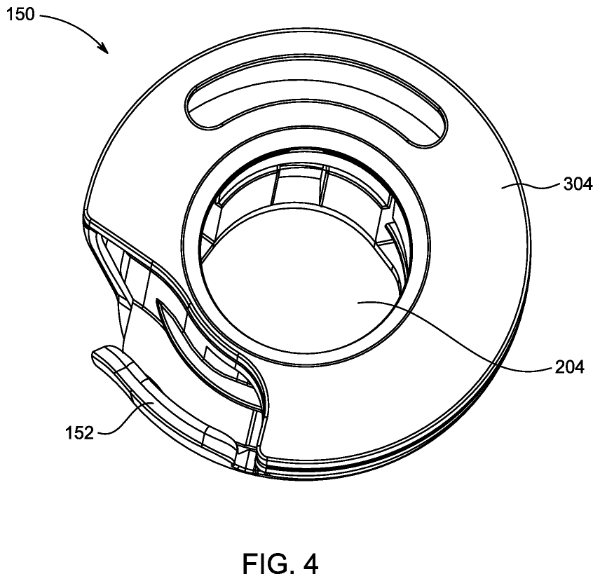

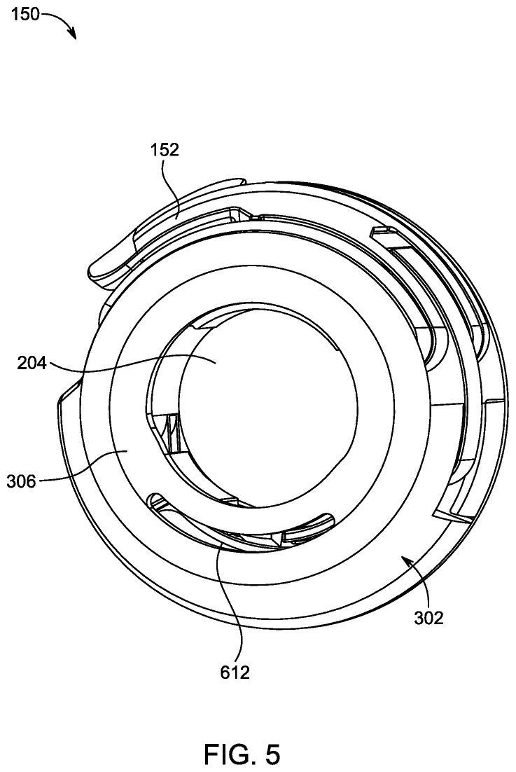

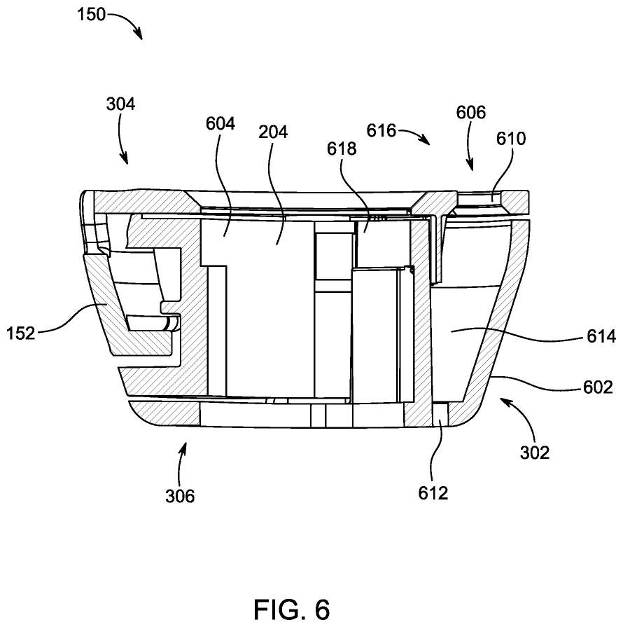

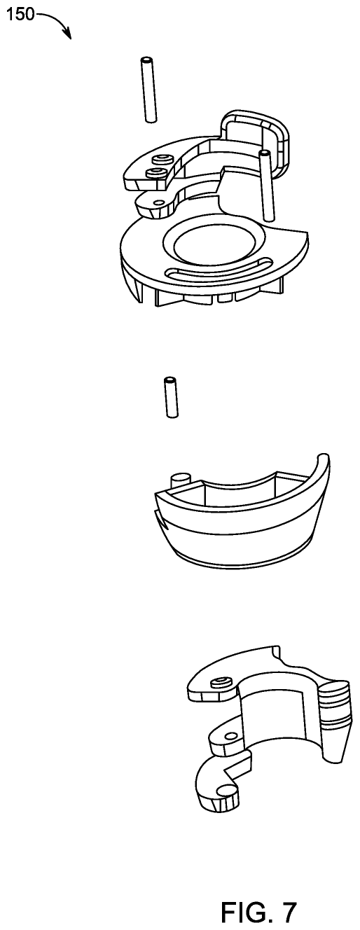

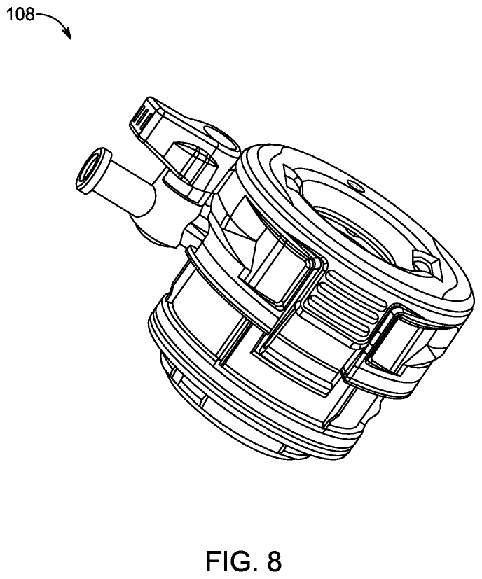

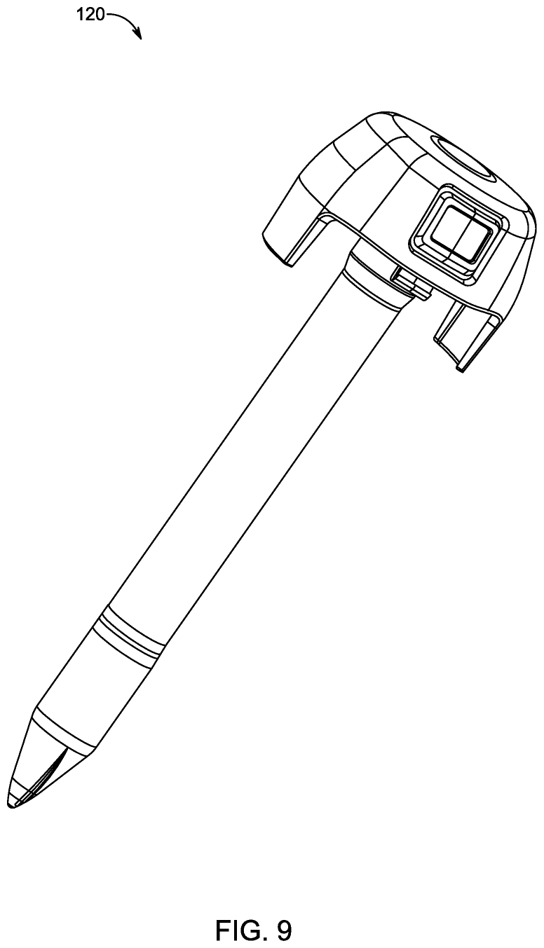

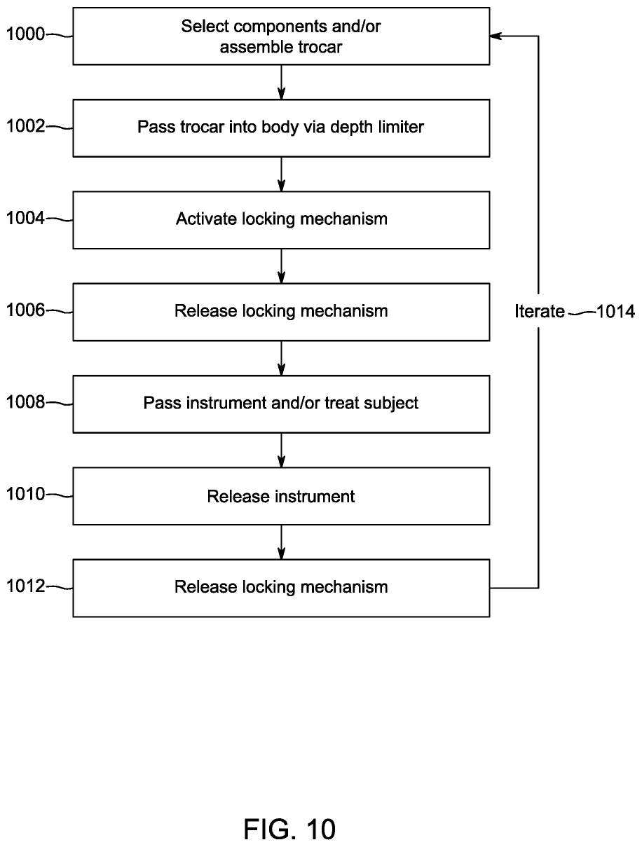

Reference is now made to FIG. 1, which is a schematic of a trocar assembly 102 that includes a depth limiter 150, in accordance with some embodiments of the present invention. Reference is also made to FIG. 2, which is a perspective view of depth limiter 150, in accordance with some embodiments of the present invention. Reference is also made to FIG. 3, which is a side view of depth limiter 150, in accordance with some embodiments of the present invention. Reference is also made to FIG. 4, which is a top view of depth limiter 150, in accordance with some embodiments of the present invention. Reference is also made to FIG. 5, which is a bottom view of depth limiter 150, in accordance with some embodiments of the present invention. Reference is also made to FIG. 6, which is a cross sectional view of depth limiter 150, in accordance with some embodiments of the present invention. Reference is also made to FIG. 7, which is an exploded view of depth limiter 150, in accordance with some embodiments of the present invention. Reference is also made to FIG. 8, which is a schematic of an exemplary sealing element 108 of trocar assembly 102, in accordance with some embodiments of the present invention. Reference is also made to FIG. 9 is a schematic of an exemplary obturator 120 of trocar assembly 120, in accordance with some embodiments of the present invention. Reference is also made to FIG. 10, which is a flowchart of an exemplary method of treating a subject using a depth limiter, in accordance with some embodiments of the present invention.

Referring now back to FIG. 1, a trocar assembly 102 includes a depth limiter component 150 placed along a shaft of a trocar cannula 110.

An exemplary trocar cannula 110 is described for example, with reference to U.S. Patent Provisional Patent Application entitled “TROCAR INCLUDING CANNULA”, Attorney Docket No. 94012, the contents of which are incorporated herein by reference in their entirety.

Trocar assembly 102 may further include an obturator 120. Obturator 120 may be bladed or non-bladed. Obturator 120 may include a piercing tip 122 which is used to pierce the skin of the subject until a cavity is entered, for example, the abdominal cavity. After penetration into the abdominal cavity is complete, obturator portion 120 may be removed from trocar cannula 110. After the obturator portion is removed, any number of surgical instruments such as, for example, a tissue fastening instrument can be inserted through the cannula 110 of the trocar assembly 102 to perform a surgical procedure.

Trocar assembly 102 may include a trocar sleeve 104 that includes a trocar housing 106 and trocar cannula 110. Obturator 120 may be designed to engage with housing 106.

Trocar assembly 102 may include a sealing element 108 designed to engage trocar housing 106. Sealing element 108 may be located within obturator 120, and/or within housing 106, and/or other designs may be implemented.

Depth limiter 150 includes a lumen sized and shaped to accommodate trocar cannula 110, and a locking mechanism 152 that sets trocar cannula 110 in a fixed position within the lumen, and/or that sets depth limiter 150 at a fixed position along trocar cannula 110. The internal diameter of the lumen of depth limiter 150 is slightly larger than the outer diameter of trocar cannula 110, to enable sliding depth limiter 150 along a length of trocar cannula 110.

Locking mechanism 152 may be reversible, for releasing trocar cannula 110 from the fixed position. Once locking mechanism 152 is released, trocar cannula 110 may be removed entirely from the lumen and from depth limiter 150, and/or trocar cannula 110 may be repositioned within the lumen of depth limiter 150. For example, depth limiter 150 is moved proximally or distally along trocar cannula 110. Once depth limiter 150 has been repositioned, locking mechanism 152 may be re-activated to fix depth limiter 150 at the new position along trocar cannula 110.

Optionally, depth limiter 150 is made from a biocompatible material that is resistant to autoclaving, enabling multiple wash and sterilization cycles (e.g., over 100, or 500, or 1000, or other values), and/or designed for multi-use, for example, polyetheretherketone (PEEK), and polyphenylsulfone (PPSU). Alternatively or additionally, depth limiter 150 is made from a biocompatible material designed for disposable and/or single use.

Depth limiter 150, which fixes cannula 110 in place once locking mechanism 152 has been activated, prevents cannula 110 from sliding deeper into the body of the patient (which may cause injury by contact with internal tissues) and/or prevents cannula 110 from sliding out of the body of the patient (which may complicate the procedure, cause gas leakages out of the body, and/or cause contamination).

Exemplary dimensions of depth limiter 150 include:
Internal diameter of the lumen of the depth limiter. The internal diameter is sized for providing an annular space between an external diameter of a trocar cannula passed through the lumen. The internal diameter of the lumen is, for example, 8.5 mm or 9 mm, or 9.3 mm or 9.5 mm, or 10 mm, for a trocar cannula having an external diameter of 5 mm. The internal diameter of the lumen is, for example, 15.5 mm or 16 mm, or 16.5 mm, or 16.9 mm, or 17.5 mm or 18 mm, for a trocar cannula having an external diameter of 12 mm.
The external diameter of the depth limiter is in the range of 21.8-30.0 mm, or 20.0 mm-32.0 mm, for a trocar cannula having an external diameter of 5 mm. The external diameter of the depth limiter is in the range of 30-38 mm, or 28 mm-40 mm, for a trocar cannula having an external diameter of 12 mm.
The thickness of the depth limiter, i.e., dimension measured from a distal end to a proximal end along a long axis though the lumen, is 18-22 mm, or 18 mm, or 20 mm, or 22 mm, regardless of the expected external diameter of the trocar cannula, for example, for external diameters of 5 mm and 12 mm of the trocar cannula.

Dimensions of the depth limiter may be selected for maintaining the trocar assembly, i.e., including cannula of the trocar sleeve, in a substantially upright position, optionally when the body cavity (e.g., abdomen) has been inflated. The trocar assembly with depth limiter placed thereon is maintained in the upright position while the trocar cannula is piercing the skin and in the body of the subject. The depth limiter maintains the trocar assembly in a substantially upright position, when the trocar assembly is not being supported by an external entity such as a human operator and/or robotic arm. Optionally, the depth limiter is sized and/or shaped for maintaining the trocar assembly in a substantially upright position while an instrument is passed through the trocar assembly. The instrument is located at the distal end region of a long rod that is passed into the body of the subject via the trocar cannula. Without the depth limiter, letting go of the trocar assembly results in the trocar assembly falling from the previously held substantially upright position to the side. The tilt in the trocar assembly to the side may damage internal organs, and/or place the instrument located in the trocar assembly outside of a sterile field which may contaminate the instrument. The tilt to the side of the trocar cannula may damage the skin and/or other tissues at the point of access into the body.

The dimensions of the depth limiter may be selected for maintain the position of the trocar sleeve, including trocar cannel, optionally with the rod of the instrument passing through the trocar sleeve, within a position range from substantially perpendicular to skin of the subject, to over about 30, or 45, or 60, or 74 degrees from a surface of the skin. The angle may be measured at the access point into the body where the trocar cannula is located, formed between a long axis of the trocar cannula and a plane tangential to the skin. Examples of selected dimensions include: a thickness (e.g., difference between the internal diameter of the lumen and the external diameter) of the depth limiter, and/or a large enough surface area of a distal surface of the depth limiter that contacts the skin to prevent or reduce tilt. In another example, a height of the depth limiter (e.g., distance between distal surface and proximal surface of the depth limiter) is sufficient to support the trocar sleeve, and/or trocar assembly, optionally when the instrument is passed therein, to withstand torque applied by the trocar sleeve, and/or trocar assembly, and/or rod of the instrument. In yet another example, the difference between the internal diameter of the lumen of the depth limiter and the external diameter of the cannula may be sufficiently small such that the walls of the internal lumen provide sufficient support to the trocar sleeve cannula to withstand torque applied by an instrument passed through.

The locking mechanism may be designed to apply sufficient pressure to the cannula to withstand torque applied by the rod of the instrument, for maintaining the rod in the substantially upright position.

Optionally, the lumen of depth limiter 150 includes one or more guides that engages the trocar cannula 110 at preselected location(s). For example, the lumen includes one or more rails designed to engage one or more depressed channels on the surface of cannula 110. In another example, the interior surface of the lumen has a non-circular shape that is designed to engage a corresponding shape of an outer surface of trocar cannula 110, for example, a square, an oval, and a four leaf clover. The guide(s), when engaged, prevent rotation of cannula 110 within the lumen of the depth limiter 150, and/or help to lock cannula 110 within the depth limiter 150.

Optionally, cannula 110 includes one or more raised features 130 on its outer surface that are designed to be engaged by the locking mechanism, for example, spaced apart elevated rings where the lock includes a C-shaped element that falls between the elevated rings, or ratchets where the locking mechanism 152 includes a pawl designed to allow easy removal of the cannula but prevent pushing the cannula deeper unless a leaver is pressed to release the pawl. Optionally, a distal end 132 of cannula excludes raise features 130, to prevent the user from fixing depth limiter 150 at a position that is too low along cannula 110, where depth limiter 150 may not be able to properly support and/or engage cannula 110. In another exemplary implementation, raise features 130 may be set at varying diameters, for example, sequentially increasing, or sequentially decreasing. For example, to enable preselection of a maximum depth, which may prevent distal pushing of the cannula and/or may enable removal. For example, the locking mechanism is set on a first diameter of the cannula 110. Proximally along cannula 110, the diameter increases, which prevents pushing cannula 110 distally into the body of the subject. Locking mechanism 152 may be preset to the first diameter before cannula 110 is inserted into the lumen of depth limiter 150, which enables the user to blindly insert cannula 110 at the predefined depth by inserting cannula 110 into the preset locking mechanism 152 of depth limiter 150.

Optionally, a kit that includes the depth limiter and one or more additional components is provided. Examples of kits include one or a combination of the following:
Multiple depth limiters of the same size.
Multiple depth limiters having lumens with different diameters. The different diameters are designed for accommodating trocar cannulas with different diameters. For example, one or more depth limiters with an internal lumen diameter of 5 millimeters, and one or more depth limiters with an internal lumen diameter of 12 millimeters.
One or more trocar cannula, of the same and/or different dimensions. The trocar cannula are sized to fit within the depth limiter. Exemplary cannula are described, for example, with reference to the 94012 application.
One or more trocar sleeves that include a trocar housing and a trocar cannula. The trocar sleeve(s) may be of the same and/or different dimensions.
One or more sealing elements, for example, designed to engage the trocar housing. The sealing elements are of the same and/or different sizes, according to other corresponding components.
One or more obturators designed to fit in a lumen of the trocar sleeve. The outer dimension of the obturator is designed to be slightly less than the inner dimension of the lumen of the trocar sleeve, to enable sliding the obturator in and out of the lumen of the trocar sleeve. For example, a clearance between the outer diameter of the obturator and an inner diameter of the lumen of the trocar sleeve is about 0.2 millimeters, or about 0.3, or about 0.4, or about 0.5 millimeters, or other values.

Referring now back to FIG. 2, an exemplary implementation of locking mechanism 152 of depth limiter 150 is shown. It is to be understood that the depicted implementation is exemplary and not necessarily limiting, as other locking mechanisms may be used.

Depth limiter 150 includes a lumen 204 into which the trocar cannula is inserted, and to which locking mechanism 152 is applied.

Locking mechanism 152 may be set and/or released by a lever 202. Pressing the lever 202 inwards towards

Optionally, locking mechanism 152 includes a pressure element designed to apply mechanical pressure for setting the trocar cannula in the fixed position by friction. For example, pressing lever 202 pushes an element against the cannula that applies the pressure. Releasing lever 202 releases the element and the pressure. For example, locking mechanism 152 includes lever 202 connected to a C shaped pin. The C shaped pin is designed to engage the trocar cannula and apply mechanism pressure to the trocar cannula in response to a pressure applied to the lever 202.

Alternatively or additionally, locking mechanism 152 engages with the trocar cannula to prevent movement of the trocar cannula, for example, pressing lever 202 pushes a movable protruding element into an indentation in the wall of the cannula. When the protruding element is engaged in the indentation, the cannula cannot be moved.

Other exemplary implementations of locking mechanism 152 are described herein, for example, with reference to FIG. 1.

A proximal aperture 610 is described herein, for example, with reference to FIG. 6.

Referring now back to FIG. 3, depth limiter 150 may include an outer surface 302 designed to reduce or prevent harm to a subject.

Optionally, depth limiter 150 may include a tapered outer surface 302, that may include an outer diameter that decreases from a proximal surface 304 of depth limiter 150 towards a distal surface 306 of depth limiter 150. The tapering of outer surface 302 may be designed to increase the total surface area that comes into contact with skin of the subject in use. During a procedure, depth limiter 150 is positioned over skin of the subject. With a low amount of pressure applied to depth limiter 150 (e.g., no external pressure such as by the user pressing), distal surface 306 of depth limiter 150 contacts the skin of the subject. When additional pressure is applied to depth limiter 150 (e.g., the user pressing down, passing instruments, stitching the trocar to the skin), distal surface 306 presses down into the skin, and surrounding nearby skin comes into contact with outer surface 302. With increasing pressure applied to depth limiter 150 which pushes depth limiter 150 deeper into the skin surface, additional portions of outer surface 302 of depth limiter 150 comes into contact with more skin. The total surface area of depth limiter 150 contacting skin increases with increasing pressure and depth, which reduces the pressure applied by depth limiter 150 on the skin. The reduction in pressure on the skin by the larger surface area of depth limiter 150 may reduce or prevent damage to the skin from pressure, for example, pressure ulcers, bruises, and the like. In contrast, consider a non-tapered, substantially parallel (i.e., equal diameter) cross sectional diameter of the depth limiter, in which case, increased pressure on depth limiter 150 increases the pressure applied by the limited distal surface 306 on the skin, which may lead to injury.

Surface 302 and/or 306 may be smooth, for preventing or reducing damage to the skin. The smoothness may allow for efficient cleaning and/or autoclaving for repeated uses of the depth limiter.

A smooth round surface 308 may provide a transition that connects between an edge of distal surface 306 and tapered outer surface 302. Round surface 308 is designed to distribute pressure and/or avoid pressure points on the skin.

Referring now back to FIG. 4, a top view of depth limiter 150, showing proximal surface 304, is presented.

Referring now back to FIG. 5, a bottom view of depth limiter 150, showing distal surface 306, is presented. A distal aperture 612 is described herein, for example, with reference to FIG. 6.

Referring now back to FIG. 6, a cross sectional view of depth limiter 150 is presented. Depth limiter includes a housing 602 that includes locking mechanism 152. Housing 602 has an inner surface 604 that defines lumen 204. Housing 602 may further include outer surface 302 (also referred to as an external lateral surface), proximal surface 304, and distal surface 306. Housing 602 has a thickness 606 defined between inner surface 604 and external lateral surface 302.

Housing 602 includes one or more apertures providing fluid communication between an internal cavity 614 of housing 604 and an external environment 616. Optionally, housing 602 includes one or more distal apertures 612 located on distal surface 306 and/or one or more proximal apertures 610 located on proximal surface 304. Optionally, at least two different apertures are provided. Apertures enable washing fluids to enter cavity 614 for washing out debris, and/or for autoclaving heat to enter cavity 614 for sterilization, for example, in implementations in which depth limiter 150 is designed for multi-use. Optionally, housing 602 includes two or more apertures to enable flow of the washing fluids from external environment 616, into cavity 614 and out into external environment 616.

Proximal aperture 610 and/or distal aperture 612 may be arc-shaped, for example, extending along an arc having a length of about 10, or 15, or 30, or 45, or, 60, or 90, or 180, or 10-180, or 30-60 degrees, or other values. The arc shape provides a large area of the aperture, enabling a sufficient amount of washing fluid to enter and/or providing a large exit port to allow the washing fluids and/or debris to exit. The arc shape provides a large port for sterilization of internal cavity 614.

Housing 602 may include one or more lumen apertures 618 located along inner surface 604 of housing 602 that defines lumen 204. Lumen aperture(s) 618 are in fluid communication with other aperture(s) of housing 602 which are in fluid communication with external environment 616, for example, proximal aperture 610 and/or distal aperture 612. The lumen apertures help wash out the surface of the lumen and/or provide additional entrance and/or exit ports for washing internal cavity 614 and/or help sterilize internal cavity 614.

Optionally, moving components of locking mechanism 152 are disposed within internal cavity 614 of the housing 602 and in fluid communication with external environment 616. Placing the moving components in internal cavity 614 enables washing and/or sterilizing the moving components.

Referring now back to FIG. 7, which presents an exploded view of components of depth limiter 150.

Referring now back to FIG. 8, exemplary sealing element 108 may be assembled as part of trocar assembly 102, and/or include in one or more kits described herein.

Referring now back to FIG. 9, exemplary obturator 120 may be assembled as part of trocar assembly 102, and/or include in one or more kits described herein.

Referring now back to FIG. 10, at 1000, suitable components are selected for assembling the trocar, optionally from the kit described herein. For example, sizes of components are selected according to the size of the subject. The trocar is assembled from the selected components. At 1002, a trocar cannula is passed into a body of a subject via a lumen of a depth limiter. The trocar cannula may be passed via an incision and/or perforation of the skin. The trocar cannula may be inserted into a cavity of the body, for example, abdomen, chest, joint, and the like.

The cavity may be inflated with a gas, to separate the skin from the underlying organs to provide space to view and/or maneuver surgical instruments.

At 1004, a locking mechanism of the depth limiter is activated for setting the trocar cannula at a selected fixed position.

The locking mechanism may be activated single-handedly following trocar insertion, optionally while the user (e.g., surgeon) holds the cannula in the other hand, where the cannula is pre-loaded into the depth limiter.

Alternatively, the order of 1002 and 1004 is reversed. The locking mechanism of the depth limited may first be activated to set the trocar cannula at the selected fixed position, prior to insertion into the body of the patient. The trocar cannula, engaged with the depth limiter via the locking mechanism, may then be inserted into the body. Placing the depth limiter on the cannula prior to insertion into the body of the patient ensures that the cannula does not enter the body of the patient deeper than set by the depth limiter.

At 1006, the locking mechanism may be released. The position of the trocar cannula may be adjusted relative to the depth limiter. The trocar cannula may be pushed deeper into the body, or retracted from the body. The locking mechanism may be re-activated for fixing the cannula at the new selected position.

At 1008, an instrument for treating tissue in the body of the patient may be passed via the trocar cannula secured at the selected fixed position by the locking mechanism of the depth limiter. The subject may be treated using the instruments.

At 1010, the instrument may be let go, for example, by a user that was holding the instrument. The depth limiter may be designed to support the instrument at a substantially upright position, preventing tipping of the instrument to the side, as described herein.

Alternatively, the trocar sleeve without instrument passed through, is let go. The depth limiter may be designed to support the trocar sleeve at the substantially upright position, preventing tipping of the trocar sleeve to the side, as described herein.

The trocar sleeve may be let go after inflation of the body cavity.

At 1012, the locking mechanism is released.

The locking mechanism may be activated single-handedly following trocar insertion, optionally while the user (e.g., surgeon) holds the cannula in the other hand.

The trocar cannula is removed from the lumen of the depth limiter.

At 1014, one or more features described with reference to 1002-1012 are iterated, for example, for different trocars, such as for placing multiple trocars at different locations on the body of the subject to provide multiple access points for multiple instruments. The features may be iterated, for example, for adjusting the depth of the trocar cannula.

It is expected that during the life of a patent maturing from this application many relevant trocars will be developed and the scope of the term trocar is intended to include all such new technologies a priori.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain

What is claimed is:

1. A kit for minimally invasive surgery, comprising:
a trocar cannula; and
a depth limiter component comprising:
a lumen sized and shaped to accommodate a trocar cannula, wherein an external diameter of the trocar cannula is sized to correspond to an internal diameter of the lumen,
a locking mechanism that sets the trocar cannula in a fixed position within the lumen,
a housing that includes the lumen and the locking mechanism, and
at least one aperture of the housing in fluid communication between an internal cavity of the housing and an external environment external to the housing.

2. The kit of claim 1, wherein the locking mechanism comprises a pressure element designed to apply mechanical pressure for setting the trocar cannula in the fixed position by friction.

3. The kit of claim 2, wherein the locking mechanism comprises a lever connected to a C shaped pin, the C shaped pin designed to engage the trocar cannula and apply mechanism pressure to the trocar cannula in response to a pressure applied to the lever.

4. The kit of claim 1, wherein the locking mechanism comprises a movable protruding element designed to engage an indentation of the trocar cannula.

5. The kit of claim 1, wherein the depth limiter further includes a distal surface that is substantially flat and smooth, for contacting skin of a subject surrounding an entry point of the trocar cannula.

6. The kit of claim 1, wherein the depth limiter further includes a tapered outer surface that is substantially smooth, that tapers outwards from an edge of a distal surface.

7. The kit of claim 6, wherein the depth limiter further comprises a smooth round surface for connecting between an edge of the distal surface and the tapered outer surface.

8. The kit of claim 1, wherein the cavity of the housing is located in a thickness between the lumen and an external lateral surface.

9. The kit of claim 1, wherein the at least one aperture of the housing comprises at least one first aperture located on a distal surface of the housing and at least one second aperture located on a proximal surface of the housing.

10. The kit of claim 9, wherein at least one of the first aperture and the second aperture are arc-shaped.

11. The kit of claim 1, wherein the housing comprises at least one third aperture located along a surface of the housing forming the lumen, the at least one third aperture in fluid communication with the at least one aperture of the housing.

12. The kit of claim 1, wherein moving components of the locking mechanism are disposed within the internal cavity of the housing and in fluid communication with the external environment.

13. The kit of claim 1, wherein the locking mechanism is reversible, for releasing the trocar cannula from the fixed position, for at least one of: removal of the trocar cannula from the lumen, and insertion of the trocar cannula further into the lumen.

14. The kit of claim 1, wherein dimensions of the depth limiter are selected for maintaining a trocar cannula in a substantially upright position that is approximately perpendicular to skin of a subject, while the trocar cannula is fixed in position in the depth limiter, and when the trocar cannula is not being supported by an external entity.

15. The kit of claim 14, wherein the dimensions of the depth limited are selected for maintaining an instrument in the substantially upright position that is approximately perpendicular to skin of the subject, when the instrument is inserted through the trocar cannula piercing the skin into the body of the subject, and when the instrument is not being supported by the external entity.

16. The kit of claim 1, wherein the depth limiter component is made from a biocompatible material that is resistant to autoclaving, designed for multi-use.

17. The kit of claim 1, wherein the lumen includes a first guide element set for engaging a corresponding second guide element of the trocar cannula, wherein the trocar cannula is insertable into the lumen by engaging the first guide element with the second element, and the trocar cannula is prevented from being inserted into the lumen when the first guide element does not engage the second guide element.

18. The kit of claim 17, wherein the first guide element engaging the second guide element prevent rotation of the trocar cannula within the depth limiter.

19. The kit of claim 1, further comprising a plurality of depth limiters having lumens with a plurality of different diameters for accommodating trocar cannulas with different diameters.

20. The kit of claim 1, further comprising a trocar sleeve that includes a trocar housing and the trocar cannula.

21. The kit of claim 1, further comprising a sealing element designed to engage the trocar housing.

22. A method of performing minimally invasive surgery, comprising:
passing a trocar cannula into a body of a subject via a lumen of a depth limiter; and
activating a locking mechanism of the depth limiter for setting the trocar cannula at a selected fixed position,
wherein instruments for treating tissue in the body of the patient are passed via the trocar cannula secured at the selected fixed position by the locking mechanism of the depth limiter.

23. The method of claim 22, further comprising releasing the locking mechanism, and at least one of: adjusting the position of the trocar cannula relative to the depth limiter and resetting the locking mechanism, and removing the trocar cannula from the lumen of the depth limiter.
